(19) United States
(12) Patent Application Publication
(10) Pub. No.: US 2002/0060180 A1
(43) Pub. Date: May 23, 2002

(54) Hemodialysis apparatus

(76) Inventor: Takashi Sugisaki, Tokyo (JP)

Correspondence Address: Michael D. Bednarek, SHAWPITTMAN, 2300 N Street, N.W., Washington, DC, 20037-1128, US

(21) Appl. No.: 09/809,183

(22) Filed: Mar. 16, 2001

(30) Foreign Application Priority Data
Sep. 28, 2000 (JP) 2000-295410

(51) Int. Cl. B01D 15/00

(52) U.S. Cl. 210/263; 210/660

(57) ABSTRACT

A novel and useful hemodialysis apparatus which can protect dialysis patients from hyperphophataemia. A phosphorus adsorbent is interposed in an extracorporeal blood circuit for dialysis. The blood taken from the body of a dialysis patient is subjected to dialysis as it flows through the extracorporeal blood circuit. At the same time, phosphorus is adsorbed and removed from the blood by the phosphorus adsorbent, thereby preventing the dialysis patients from experiencing hyperphosphataemia. As the phosphorus adsorbent adsorbs the phosphorus from the blood flowing through the extracorporeal blood circuit, it never enters the body of the patient and therefore does not cause any adverse effects.

### BACKGROUND OF THE INVENTION

1. Field of the Invention The present invention relates to a hemodialysis apparatus.

2. Description of the Related Art

Hemodialysis, as one of the therapies used to treat chronic renal insufficiency (end-stage renal disease patients), is a medical process in which the blood of a patient is continuously taken out from the body, fed through a dialyzer, where waste products in the blood are removed, and purified blood is continuously fed back to the body. The hemodialysis apparatus therefor, is generally provided with an extracorporeal blood circuit for continuously taking the blood out of the patient's body and for returning the blood continuously to the patient's body, and a dialyzer which is interposed in the extracorporeal blood circuit. In the dialyzer, materials migrate between the blood and the dialysis liquid through a dialysis membrane, according to the principle of diffusion and the like, waste products are removed from the blood, and necessary materials are supplied to the blood.

Patients who undergo dialysis treatment may exhibit hyperphosphataemia since they accumulate phosphorus in the body due to a decrease in the excretory function of the kidney. Of course, the phosphorus intake of the dialysis patients can be controlled by alimentary therapy. However, since phosphorus intake is correlated with the protein intake which is essential to maintain a living body, too severe a restriction of the phosphorus intake will naturally result in an insufficient protein intake and will lead to malnutrition in the long run.

Therefore, in addition to dietary control, a method of preventing, by spontaneous excretion, the excessive intake of the phosphorus into the body by using a so-called phosphorus adsorbent drug has been studied. The phosphorus adsorbent combines with the ingested phosphorus in the body and prevents the phosphorus from being absorbed into the blood.

As for the phosphorus adsorbent, various phosphorus adsorbents with different compositions have been examined. However, none of them are free from adverse effects once they are taken in the body.

### SUMMARY OF THE INVENTION

The present invention has been achieved in consideration of these circumstances, and accordingly, it is an object of the present invention to provide a novel and useful hemodialysis apparatus which can protect the dialysis patients from hyperphosphataemia.

In order to solve the above-mentioned problems, a hemodialysis apparatus according to the present invention is provided with an extracorporeal blood circuit for dialysis and a phosphorus adsorbent for adsorbing the phosphorus in the blood flowing through the extracorporeal blood circuit.

According to the present invention, the blood taken out from the body of the patient for dialysis is allowed to flow through the extracorporeal blood circuit for dialysis and while it flows therethrough, the phosphorus is adsorbed by the phosphorus adsorbent and removed from the blood. In the present invention, as the phosphorus adsorbent has an excellent phosphorus removing ability, a larger amount of phosphorus can be removed from the blood and dialysis patients can be prevented from experiencing hyperphosphataemia. Moreover, since the phosphorus adsorbent adsorbs the phosphorus from the blood flowing through the extracorporeal blood circuit, the phosphorus adsorbent does not enter the body of the patient, and therefore it does not cause any adverse reactions.

In the hemodialysis apparatus, an activated charcoal, which is in contact with the blood flowing through the extracorporeal blood circuit can be provided on the downstream side of the phosphorus adsorbent in the extracorporeal blood circuit. Such an arrangement is desirable as, in the event that some impurities from the phosphorus adsorbent are mixed into the blood, those impurities are captured by the activated charcoal due to its excellent absorbing ability.

A hemodialysis apparatus which is provided with a calcium carbonate-containing material which is brought into contact with the blood flowing through the extracorporeal blood circuit to supply calcium ion into the blood is desirable, because the calcium carbonate-containing material can supply calcium ion to the blood, thereby also preventing calcium deficiency in the dialysis patients.

A use of a calcium carbonate-containing material also containing trace mineral elements necessary for the human body is preferable.

A coral processed product can be used for the calcium carbonate-containing material. As coral has a high calcium content and a large contact area with the blood due to its large surface area, it has a good calcium-supplying ability. In addition, coral contains many kinds of trace mineral elements which are necessary for the human body, for example, Na, Mg, K, P, Fe, Cl, S, Si, Al, Sr, Cr, Co, Ni, Mn, Cu, Zn, Mo and so on. Therefore, the use of coral is more desirable since these trace mineral elements ion can also be supplied to the blood.

Installation of the coral processed product on the downstream side of the phosphorus adsorbent in the extracorporeal blood circuit is even more desirable, since the blood can be purified by the coral processed product having a large surface area.

Another embodiment of the hemodialysis apparatus according to the present invention is provided with an extracorporeal blood circuit for dialysis, and at least one of a phosphorus adsorbent to adsorb the phosphorus in the blood flowing through the extracorporeal blood circuit and a coral processed product which supplies calcium ion and other trace mineral elements ion to the blood flowing through the extracorporeal blood circuit. If the phosphorus adsorbent is provided, the same results as those obtained with the hemodialysis apparatus described the corresponding description above can be obtained and, when the coral processed product is provided, the same results as those obtained with the hemodialysis apparatus described in the corresponding description above can be obtained.

The present invention will hereinafter be described with one preferred embodiment thereof with reference to the attached FIGURE.

### BRIEF DESCRIPTION OF THE DRAWING

FIG. 1 is a diagram of a hemodialysis circuit including a hemodialysis apparatus according to one embodiment of the present invention.

### DESCRIPTION OF THE PREFERRED EMBODIMENT

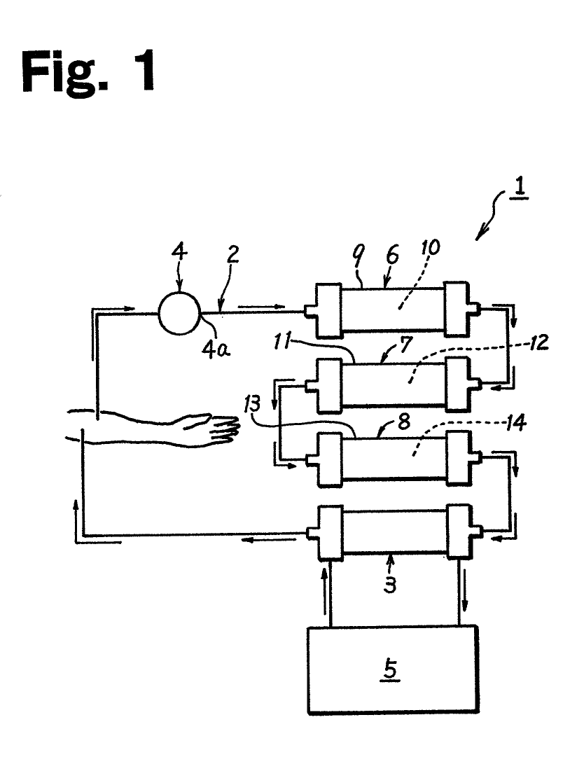

Referring now to FIG. 1, the blood taken out for dialysis from the body (arm) of a dialysis patient flows through an extracorporeal blood circuit 2 for dialysis and as it passes therethrough it is subjected to dialysis by a dialyzer 3 provided in the circuit 2 and then it is returned to the body of the dialysis patient. The circulation of the blood is controlled by a blood pump 4 provided in the extracorporeal blood circuit 2.

It is possible to use a coil type dialyzer or a laminate type dialyzer for the dialyzer 3. However, the use of a hollow yarn type dialyzer, which is currently the most widely employed, is desirable due to its excellent dialytic performance.

A dialysis liquid is continuously supplied from a dialysis liquid supply apparatus 5 (which is conventionally known) to the dialyzer 3, and the dialysis liquid is brought into contact with the blood via a dialysis membrane (not shown in the Fig.) in the dialyzer 3, and is then returned to the dialysis liquid supply apparatus 5. In the dialyzer 3, materials migrate between the blood and the dialysis liquid by the principle of diffusion, etc., waste products such as uremic materials are removed from the blood, and necessary materials are supplied to the blood.

In this embodiment, the extracorporeal blood circuit 2 is provided with a phosphorus adsorbent part 6, a calcium supply part 7, and an activated charcoal part 8, in that order, from the upstream side to the downstream side of the blood flow between the discharge side 4a of the blood pump 4 and the dialyzer 3.

The phosphorus adsorbent part 6 is provided with a phosphorus adsorbent container 9 and a phosphorus adsorbent 10 contained in the phosphorus adsorbent container 9. The phosphorus adsorbent may have various compositions. However, the phosphorus adsorbent used herein should have good phosphorus adsorption ability and should not release any undesirable components into the blood even when it comes into contact with the blood. For example, a polycation polymer developed by GelTex U.S.A. having the following molecular structure can be used. This phosphorus adsorbent adsorbs phosphorus in exchange for HCl.

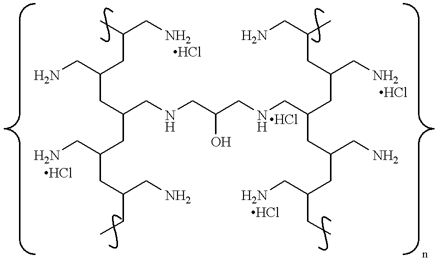

The phosphorus adsorbent 10 comes into contact with the blood flowing into the phosphorus adsorbent container 9, and adsorbs and removes the phosphorus contained in the blood due to its excellent phosphorus adsorbing ability, and thus, it can protect the dialysis patients from hyperphosphataemia. Furthermore, since the phosphorus adsorbent 10 comes into contact with the blood to adsorb the phosphorus after the blood has been taken out of the body, it does not enter the body of the patient, and therefore it doesn't cause any adverse effects. The blood from which the phosphorus is removed in the phosphorus adsorbent part 6 flows into the calcium supply part 7.

The calcium supply part 7 is provided with a container 11 containing a calcium carbonate-containing material 12. The calcium carbonate-containing material 12 supplies calcium ion from its surface to the blood when it is in contact with the blood. There are various kinds of calcium carbonate-containing materials, however, a desirable calcium carbonate-containing material for use in this embodiment is one in which, in addition to its high calcium ion content, when the calcium carbonate-containing material comes into contact with the blood, the calcium ion will be sufficiently eluted in the blood. Moreover the use of a calcium carbonate-containing material also containing trace mineral elements which are necessary for the human body is preferable, because these trace mineral elements ion can also be supplied to the blood. For example, coral calcium granules which are produced from weathered hermatypic coral, and other coral processed products produced from coral as the raw material, can be used. As coral not only contains a large amount of calcium, but also has a large surface area, it has a large contact area with the blood, and can sufficiently supply the calcium to the blood. Besides, the use of coral is more desirable, because coral contains many kinds of balanced trace minerals, which are necessary for the human body, for example, Na, Mg, K, P, Fe, Cl, S, Si, Al, Sr, Cr, Co, Ni, Mn, Cu, Zn, Mo and so on. By using of coral, not only the calcium ion but also those trace mineral elements ion can be supplied to the blood.

It is still more desirable that the coral processed product 12 be provided on the downstream side of the phosphorus adsorbent 10 in the extracorporeal blood circuit 2, as it is in the present embodiment, so that even if some impurities from the phosphorus adsorbent 10 are mixed in the blood, the coral processed product 12 having a large surface area can purify the blood well.

The blood, having received calcium ion and other trace mineral elements ion in the calcium supply part 7, flows into the activated charcoal part 8.

The activated charcoal part 8 is provided with an activated charcoal container 13 and activated charcoal 14 contained in the activated charcoal container 13. The activated charcoal 14 purifies the blood when it is in contact with its large surface area. It is particularly desirable that the activated charcoal part 8 be provided on the downstream side of the blood flow from the phosphorus adsorbent 10, as it is in the present embodiment, so that even if impurities from the phosphorus adsorbent 10 are mixed in the blood, the activated charcoal 14 can capture the impurities due to its excellent adsorbing ability.

The blood purified in the activated charcoal part 8, then flows into the dialyzer 3, is subjected to dialysis in the dialyzer 3, and is returned to the body of the dialysis patient.

The above-mentioned process is continuously carried out for the dialysis period. The phosphorus can be effectively removed from the blood continuously during the dialysis period by the hemodialysis apparatus 1, calcium ion is supplied to the blood, and the hyperphosphataemia and hypocalcemia of the dialysis patients can be prevented or cured. Thereby complications arising from hyperphosphataemia and hypocalcemia can be prevented, thus helping to extend the life of the patients on dialysis.

The order of the alignment of the phosphorus adsorbent part 6, the calcium supply part 7, the activated charcoal part 8, and the dialyzer 3 in the extracorporeal blood circuit 2 is not necessarily limited to that used in the present embodiment, however, it is desirable to align them in the order used in the present embodiment with respect to the direction of the blood flow, so that the above-mentioned advantages can be achieved.

Moreover, only one of the phosphorus adsorbent part 6 and the calcium supply part 7 may be provided. When the phosphorus adsorbent part 6 is provided, phosphorus removal can be achieved by the phosphorus adsorbent 10, and when the calcium supply part 7 is provided, calcium ion supply effect can be obtained due to the calcium carbonate-containing material 12.

The present inventors have measured the phosphorus removal effect and the calcium supply effect by the hemodialysis apparatus according to the present embodiment by using dogs and monkeys. The experiments are as follows.

<Experiment 1>

Three 7-month-old, male beagles each having a body weight of 8-9 kg were used in this experiment. These dogs were fed on solid food produced by Oriental Yeast Co., Ltd.

The dogs were starved for 16 hours, then the blood was taken from the cephalic vein of each dog under anesthesia. The blood was treated with heparin (anticoagulant) and the resulting separated serum was used to measure the calcium concentration by the OCPC method and the phosphorus (inorganic phosphorus) concentration by the molybdenic acid method. The results of the measurement are shown in Table 1, row A-1.

Then the hemodialysis apparatus according to the present embodiment was immediately connected to the cephalic artery, blood was taken, and the calcium concentration and the phosphorus concentration were measured 1, 2 and 3 hours later using the same method as described above. The results of the measurement are shown in Table 1, row A-2.

TABLE 1

| | | |
|---|---|---|
| No. 1 | No. 2 | No. 3 |

Serum calcium levels (mg/dl)

| | | | | | |
|---|---|---|---|---|---|
| A-1 | | 10.1 | 9.7 | 9.2 | 9.7 ± 0.4 |
| (before | | | | | |
| dialysis) | | | | | |
| A-2 | After 1 | 10.0 | 9.8 | 9.3 | 9.7 ± 0.4 |
| (after | hour | | | | |
| Dialysis) | After 2 | 9.8 | 9.7 | 9.5 | 9.7 ± 0.2 |
| | hours | | | | |
| | After 3 | 10.2 | 9.9 | 9.7 | 9.9 ± 0.2* |
| | hours | | | | |

*P < 0.01 (Significant difference
observed)

Serum phosphorus (inorganic phosphorus) levels (mg/dl)

| | | | | | |
|---|---|---|---|---|---|
| A-1 | | 6.4 | 5.8 | 6.2 | 6.1 ± 0.2 |
| (before | | | | | |
| dialysis) | | | | | |
| A-2 | After 1 | 4.8 | 3.8 | 3.9 | 4.2 ± 0.5* |
| (after | hour | | | | |
| dialysis) | After 2 | 4.3 | 3.6 | 3.8 | 3.9 ± 0.3* |
| | hours | | | | |
| | After 3 | 4.1 | 3.2 | 3.3 | 3.5 ± 0.3* |
| | hours | | | | |

*P < 0.01 (Significant difference
observed)
Assay : Dunnett method, Kruskal-Wallis method

In this experiment, a significant increase in the serum calcium levels 3 hours after the start of the dialysis was observed. As for the serum phosphorus levels, a significant decrease was observed 1, 2 and 3 hours after the start of the dialysis.

<Experiment 2>

Male cynomolgus monkeys each having a body weight of 2-3 kg and an age of 2-3 years were used in this experiment. These monkeys were fed on simian feedstuff.

The monkeys were starved for 16 hours, then blood was taken from the cephalic vein of each monkey under anesthesia. The blood was treated with heparin (anticoagulant) and separated serum was used to measure the calcium concentration and phosphorus (inorganic phosphorus) concentration. The results of the measurement are shown in Table 2, row B-1.

Then, the hemodialysis apparatus according to the present embodiment was immediately connected to the cephalic artery and the blood was taken 1, 2 and 3 hours later and serum calcium levels and the serum phosphorus levels were measured. The results of the measurement are shown in Table 2, row B-2.

TABLE 2

| | | |
|---|---|---|
| No. 1 | No. 2 | No. 3 |

Serum calcium levels (mmol/l)

| | | | | | |
|---|---|---|---|---|---|
| B-1 | | 2.5 | 2.8 | 2.7 | 2.7 ± 0.2 |
| (before | | | | | |
| dialysis) | | | | | |
| B-2 | After 1 | 2.8 | 3.0 | 2.9 | 2.9 ± 0.1* |
| (after | hour | | | | |
| dialysis) | After 2 | 2.8 | 3.1 | 3.0 | 3.0 ± 0.1* |
| | hours | | | | |
| | After 3 | 3.0 | 3.1 | 3.2 | 3.1 ± 0.1* |
| | hours | | | | |

*P < 0.01 (Significant difference
observed)

Serum phosphorus (inorganic phosphorus) levels (mmol/l)

| | | | | | |
|---|---|---|---|---|---|
| B-1 | | 2.2 | 2.1 | 1.9 | 2.1 ± 0.2 |
| (before | | | | | |
| dialysis) | | | | | |
| B-2 | After 1 | 1.5 | 1.7 | 1.5 | 1.6 ± 0.2* |
| (after | hour | | | | |
| dialysis) | After 2 | 1.2 | 1.5 | 1.3 | 1.3 ± 0.2* |
| | hours | | | | |
| | After 3 | 1.0 | 1.3 | 1.1 | 1.1 ± 0.2* |
| | hours | | | | |

*P < 0.01 (Significant difference
observed)
Assay : Dunnett method, Behrens-Fisher method

In this experiment, a significant increase in the serum calcium levels at 1, 2 and 3 hours after the start of dialysis was observed. As for the serum phosphorus levels, a significant decrease was observed 1, 2 and 3 hours after the start of dialysis.

What is claimed is:

1. A hemodialysis apparatus comprising:
an extracorporeal blood circuit for dialysis; and
a phosphorus adsorbent for adsorbing the phosphorus in the blood flowing through said extracorporeal blood circuit.

2. The hemodialysis apparatus according to claim 1, wherein an activated charcoal which is in contact with the blood flowing through said extracorporeal blood circuit is provided on the downstream side from said phosphorus adsorbent in said extracorporeal blood circuit.

3. The hemodialysis apparatus according to claim 1, further comprising a calcium carbonate-containing material which is placed in contact with the blood flowing through said extracorporeal blood circuit to supply calcium ion to said blood.

4. The hemodialysis apparatus according to claim 3, wherein said calcium carbonate-containing material contains trace minaral elements necessary for the human body which supplies trace mineral elements ion to said blood.

5. The hemodialysis apparatus according to claim 3, in which said calcium carbonate-containing material is a coral processed product.

6. The hemodialysis apparatus according to claim 5, in which said coral processed product is provided on the downstream side from said phosphorus adsorbent in said extracorporeal blood circuit.

7. A hemodialysis apparatus comprising:
an extracorporeal blood circuit for dialysis;
at least one of a phosphorus adsorbent for adsorbing the phosphorus in the blood flowing through said extracorporeal blood circuit and a coral processed product for supplying calcium ion and trace mineral elements ion to the blood flowing through said extracorporeal blood circuit.
